(19) United States
(12) Patent Application Publication
(10) Pub. No.: US 2008/0020433 A1
(43) Pub. Date: Jan. 24, 2008

(54) Process for Producing Amino Acids

(75) Inventors: Shin-ichi Hashimoto, Hofu-shi (JP); Kouichi Terazono, Tokyo (JP); Akinori Yasuhara, Yamaguchi-shi (JP); Kazunobu Matsushita, Yamaguchi (JP)

Correspondence Address: FITZPATRICK CELLA HARPER & SCINTO, 30 ROCKEFELLER PLAZA, NEW YORK, NY, 10112, US

(73) Assignee: KYOWA HAKKO KOGYO CO,M LTD., TOKYO JAPAN (JP)

(21) Appl. No.: 10/590,705

(22) PCT Filed: Feb. 25, 2005
(86) PCT No.: PCT/JP05/03694

§ 371 Date: Jun. 14, 2007

(30) Foreign Application Priority Data
Feb. 27, 2004 (JP) 2004-053361

(51) Int. Cl. C12P 13/04; C12N 1/21; C12P 13/06; C12P 13/10; C12P 13/14; C12P 13/22; C12P 13/24; C12P 13/20; C12P 13/12; C12P 13/08; C12N 15/00

(52) U.S. Cl. 435/107; 435/106; 435/108; 435/109; 435/110; 435/113; 435/114; 435/115; 435/116; 435/252.32; 435/252.33; 435/320.1

(57) ABSTRACT

The present invention provides a microorganism obtainable by introducing a DNA coding for NADH dehydrogenase constituting a NADH dehydrogenase complex whose number of dischargeable proton molecules per electron is zero among NADH dehydrogenase complexes functioning as a proton pump in electron transport systems of aerobic bacteria, and an industrially advantageous process for producing an amino acid using the microorganism.

### TECHNICAL FIELD

The present invention relates to a process for producing an amino acid.

### BACKGROUND ART

In recent years, attempts to improve the productivity of substances such as amino acids, etc. by mutating electron transport systems of microorganisms have been reported.

By way of examples, it has been reported that productivity of an amino acids is improved either by amplifying DNA coding for energy production NADH dehydrogenase or by deleting DNA coding for energy non-production NADH dehydrogenase, of Escherichia coli (Japanese Published Unexamined Patent Application No. 17363/2002).

Further, it has been reported that productivity of riboflavin is improved by amplifying DNA coding for cytochrome bc oxidase having high ratio of energy generation efficiency in Bacillus subtilis (WO03/072785).

With respect to the influence of mutation in electron transport systems on the growth of microorganisms, Molenaar reported that destruction of DNA coding for NADH dehydrogenase of Corynebacterium glutamicum did not raise any influence on growth of the microorganism (Journal of Bacteriology, 182, p6884-6891 (2000)).

As described above, it is known that there is a possibility that the mutation in electron transport systems of microorganisms raises influence on productivity of substances by microorganisms. However, it is difficult to predict how the influence is raised.

In addition to conventional methods, further development of methods for improving productivity of substances is demanded.

### DISCLOSURE OF THE INVENTION

It is an object of the present invention to provide an industrially advantageous process for producing amino acids The present invention relates to the following (1) to (26).

(1) A process for producing an amino acid, which comprises culturing, in a medium, a microorganism obtainable by introducing a DNA coding for energy non-production NADH dehydrogenase, forming and accumulating an amino acid in a culture, and recovering the amino acid from the culture.

(2) The process according to (1), wherein the DNA coding for energy non-production NADH dehydrogenase is a DNA derived from a microorganism selected from the group consisting of microorganisms belonging to the genus Corynebacterium, Escherichia, Pseudomonas, Azotobacter, Salmonella or Lactobacillus, or a DNA which hybridizes, under stringent conditions, with a DNA having a nucleotide sequence complementary to the nucleotide sequence of the DNA.

(3) The process according to (1), wherein the DNA coding for energy non-production NADH dehydrogenase is a DNA derived from a microorganism selected from the group consisting of microorganisms belonging to the species Corynebacterium glutamicum, Corynebacterium diphtheriae, Escherichia coli, Pseudomonas fluorescens, Azotobacter vinelandii, Salmonella typhimurium or Lactobacillus plantarum, or a DNA which hybridizes, under stringent conditions, with a DNA having a nucleotide sequence complementary to the nucleotide sequence of the DNA.

(4) The process according to (1), wherein the DNA coding for energy non-production NADH dehydrogenase is a DNA having a nucleotide sequence selected from the group consisting of nucleotide sequences represented by SEQ ID NOs: 3, 5, 7, 9, 11, 13 and 15, or a DNA which hybridizes, under stringent conditions, with a DNA having a nucleotide sequence complementary to the nucleotide sequence.

(5) The process according to (1), wherein the DNA coding for energy non-production NADH dehydrogenase is a DNA coding for energy non-production NADH dehydrogenase possessed by a plasmid pCS-CGndh carried by Escherichia coli DH5α/pCS-CGndh (FERM BP-08633) or a DNA which hybridizes, under stringent conditions, with a DNA having a nucleotide sequence complementary to the nucleotide sequence of the DNA and which encodes a polypeptide having the energy non-production NADH dehydrogenase activity.

(6) The process according to (1), wherein the energy non-production NADH dehydrogenase is a polypeptide having an amino acid sequence selected from the group consisting of amino acids sequences represented by SEQ ID NOs: 4, 6, 8, 10, 12, 14 and 16, or a polypeptide comprising an amino acid sequence wherein one or more amino acid residues are deleted, substituted or added in the amino acid sequence of the polypeptide and having the energy non-production NADH dehydrogenase activity.

(7) The process according to (1), wherein the energy non-production NADH dehydrogenase is a polypeptide encoded by the DNA coding for energy non-production NADH dehydrogenase possessed by a plasmid pCS-CGndh carried by Escherichia coli DH5α/pCS-CGndh (FERM BP-08633) or a polypeptide comprising an amino acid sequence wherein one or more amino acid residues are deleted, substituted or added in the amino acid sequence of the polypeptide and having the energy non-production NADH dehydrogenase activity.

(8) The process according to any one of (1) to (7), wherein the microorganism into which the DNA coding for energy non-production NADH dehydrogenase is introduced is a microorganism selected from the group consisting of microorganisms belonging to the genus Escherichia, Corynebacterium, Brevibacterium, Arthrobacter, Aureobacterium, Cellulomonas, Clavibacter, Curtobacterium, Microbacterium, Pimerobacter or Bacillus.

(9) The process according to any one of (1) to (7), wherein the microorganism into which the DNA coding for energy non-production NADH dehydrogenase is introduced is a microorganism belonging to the genus Escherichia.

(10) The process according to any one of (1) to (7), wherein the microorganism into which the DNA coding for energy non-production NADH dehydrogenase is introduced is a microorganism belonging to the species Escherichia coli.

(11) The process according to any one of (1) to (7), wherein the microorganism into which the DNA coding for energy non-production NADH dehydrogenase is introduced is a microorganism belonging to the genus Corynebacterium.

(12) The process according to any one of (1) to (7), wherein the microorganism into which the DNA coding for energy non-production NADH dehydrogenase is introduced is a microorganism selected from the group consisting of microorganisms belonging to the species Corynebacterium glutamicum, Corynebacterium flavum, Corynebacterium lactofermentum, or Corynebacterium efficasis.

(13) The process according to any one of (1) to (7), wherein the microorganism into which the DNA coding for energy non-production NADH dehydrogenase is introduced is a microorganism belonging to the species Corynebacterium glutamicum.

(14) The process according to any one of (1) to (13), wherein the amino acid is an amino acid selected from the group consisting of L-glutamic acid, L-glutamine, L-aspartic acid, L-asparagine, L-lysine, L-methionine, L-threonine, L-arginine, L-proline, L-citrulline, L-valine, L-leucine, L-isoleucine, L-serine, L-cysteine, glycine, L-triptophan, L-thyrosine, L-phenylalanine and L-histidine.

(15) The process according to any one of (1) to (13), wherein the amino acid is an amino acid selected from the group consisting of L-glutamic acid, L-glutamine and L-lysine.

(16) A microorganism which belongs to the genus Corynebacterium, and is obtainable by introducing a DNA coding for energy non-production NADH dehydrogenase.

(17) A microorganism which belongs to the species Corynebacterium glutamicum, and is obtainable by introducing a DNA coding for energy non-production NADH dehydrogenase.

(18) The microorganism according to (16) or (17), wherein the DNA coding for energy non-production NADH dehydrogenase is a DNA derived from a microorganism selected from the group consisting of microorganisms belonging to the genus Corynebacterium, Escherichia, Pseudomonas, Azotobacter, Salmonella or Lactobacillus, or a DNA which hybridizes, under stringent conditions, with a DNA having a nucleotide sequence complementary to the nucleotide sequence of the DNA.

(19) The microorganism according to (16) or (17), wherein the DNA coding for energy non-production NADH dehydrogenase is a DNA derived from a microorganism selected from the group consisting of microorganisms belonging to the species Corynebacterium glutamicum, Corynebacterium diphtheriae, Escherichia coli, Pseudomonas fluorescens, Azotobacter vinelandii, Salmonella typhimurium or Lactobacillus plantarum, or a DNA which hybridizes, under stringent conditions, with a DNA having a nucleotide sequence complementary to the nucleotide sequence of the DNA.

(20) The microorganism according to (16) or (17), wherein the DNA coding for energy non-production NADH dehydrogenase is a DNA having a nucleotide sequence selected from the group consisting of nucleotide sequences represented by SEQ ID NOs: 3, 5, 7, 9, 11, 13 and 15, or a DNA which hybridizes, under stringent conditions, with a DNA having a nucleotide sequence complementary to the nucleotide sequence.

(21) The microorganism according to (16) or (17), wherein the DNA coding for energy non-production NADH dehydrogenase is a DNA coding for energy non-production NADH dehydrogenase possessed by a plasmid pCS-CGndh carried by Escherichia coli DH5α/pCS-CGndh (FERM BP-08633) or a DNA which hybridizes, under stringent conditions, with a DNA having a nucleotide sequence complementary to the nucleotide sequence of the DNA and which encodes a polypeptide having the energy non-production NADH dehydrogenase activity.

(22) The microorganism according to (16) or (17), wherein the energy non-production NADH dehydrogenase is a polypeptide having an amino acid sequence selected from the group consisting of amino acids sequences represented by SEQ ID NOs: 4, 6, 8, 10, 12, 14 and 16, or a polypeptide comprising an amino acid sequence wherein one or more amino acid residues are deleted, substituted or added in the amino acid sequence of the polypeptide and having the energy non-production NADH dehydrogenase activity.

(23) The microorganism according to (16) or (17), wherein the energy non-production NADH dehydrogenase is a polypeptide encoded by a DNA possessed by a plasmid pCS-CGndh carried by Escherichia coli DH5α/pCS-CGndh (FERM BP-08633) or a polypeptide comprising an amino acid sequence in which one or more amino acid residues are deleted, substituted or added in the amino acid sequence of the polypeptide and having the energy non-production NADH dehydrogenase activity.

(24) Corynebacterium glutamicum ATCC 14752/pCS-CGndh or Corynebacterium glutamicum FERM BP-1069/pCS-CGndh.

(25) Escherichia coli DH5α/pCS-CGndh (FERM BP-08633).

(26) A plasmid pCS-CGndh carried by Escherichia coli DH5α/pCS-CGndh (FERM BP-08633).

### BEST MODE FOR CARRYING OUT THE INVENTION

Energy non-production NADH dehydrogenase (hereinafter referred to also as NDH polypeptide) used in the present invention may be any polypeptide having an activity of NADH dehydrogenase constituting an NADH dehydrogenase complex, by which number of proton molecules discharged per electron is zero (hereinafter the activity is referred to as an energy non-production NADH dehydrogenase activity) among NADH dehydrogenase complexes functioning as a proton pump in electron transport systems of aerobic bacteria.

Examples of the NDH polypeptide include known NDH polypeptides derived from microorganisms belonging to the genus Corynebacterium, Escherichia, Pseudomonas, Azotobacter, Salmonella or Lactobacillus, and the like.

Microorganisms belonging to the genus Corynebacterium include microorganisms belonging to Corynebacterium glutamicum or Corynebacterium diphtheriae, and the like.

Microorganisms belonging to the genus Escherichia include microorganisms belonging to Escherichia coli, and the like.

Microorganisms belonging to the genus Pseudomonas include microorganisms belonging to Pseudomonas fluorescens, and the like.

Microorganisms belonging to the genus Azotobacter include microorganisms belonging to Azotobacter vinelandii.

Microorganisms belonging to the genus Salmonella include microorganisms belonging to Salmonella typhimurium.

Microorganisms belonging to the genus Lactobacillus include microorganisms belonging to Lactobacillus plantarum.

The NDH polypeptides derived from these microorganisms include known NDH polypeptides such as a polypeptide derived from microorganisms belonging to the genus Corynebacterium and having an amino acid sequence represented by SEQ ID NO: 4 or 6, a polypeptide derived from microorganisms belonging to the genus Escherichia and having an amino acid sequence represented by SEQ ID NO: 8, a polypeptide derived from microorganisms belonging to the genus Pseudomonas and having an amino acid sequence represented by SEQ ID NO: 10, a polypeptide derived from microorganisms belonging to the genus Azotobacter and having an amino acid sequence represented by SEQ ID NO: 12, a polypeptide derived from microorganisms belonging to the genus Salmonella and having an amino acid sequence represented by SEQ ID NO: 14 and a polypeptide derived from microorganisms belonging to the genus Lactobacillus and having an amino acid sequence represented by SEQ ID NO: 16.

A polypeptide encoded by a DNA (ndh) coding for Corynebacterium glutamicum-derived energy non-production NADH dehydrogenase possessed by plasmid pCS-CGndh carried by Escherichia coli DH5α/pCS-CGndh (FERM BP-08633)(hereinafter abbreviated as NDH polypeptide A) can also be mentioned as an NDH polypeptide.

The NDH polypeptide used in the present invention may be a polypeptide comprising an amino acid sequence in which one or more amino acid residues are deleted, substituted or added in the amino acid sequence of NDH polypeptide A or known NDH polypeptides so long as it has the energy non-production NADH dehydrogenase activity.

The polypeptide comprising the amino acid sequence in which one or more amino acid residues are deleted, substituted or added in the amino acid sequence of NDH polypeptide A or known NDH polypeptides can be obtained by introducing site-specific mutation in the DNA coding for NDH polypeptide A or known NDH polypeptides using a site-specific mutation introducing method described in Molecular Cloning, A Laboratory Manual, Third Edition, Cold Spring Harbor Laboratory Press (2001) (hereinafter abbreviated as Molecular Cloning 3rd ed.), Current Protocols in Molecular Biology, John Wiley & Sons (1987-1997) (hereinafter abbreviated as Current Protocols in Molecular Biology), Nucleic Acids Research, 10, 6487 (1982), Proc. Natl. Acad. Sci. USA, 79, 6409 (1982), Gene, 34, 315 (1985), Nucleic Acids Research, 13, 4431 (1985), Proc. Natl. Acad. Sci. USA, 82, 488 (1985) and the like.

The number of amino acid residues to be deleted, substituted or added is not particularly limited. It is the number of amino acid residues which can be deleted, substituted or added by a known method such as the site-specific mutation method. The number is from 1 to several tens, preferably from 1 to 20, more preferably from 1 to 10, further preferably from 1 to 5.

That one or more amino acid residues are deleted, substituted or added in an amino acid sequence of NDH polypeptide A or known NDH polypeptides means that one or plural amino acid residues are deleted, substituted or added in any one or plural sites of one and the same amino acid sequence. Deletion, substitution or addition may take place simultaneously.

An amino acid to be substituted or added may be a natural type or a non-natural type.

Examples of the natural-type amino acid include L-alanine, L-asparagine, L-aspartic acid, L-glutamine, L-glutamic acid, glycine, L-histidine, L-isoleucine, L-leucine, L-lysine, L-arginine, L-methionine, L-phenylalanine, L-proline, L-serine, L-threonine, L-tryptophan, L-tyrosine, L-valine, L-cysteine and the like.

Examples of amino acids which can mutually be substituted are listed below. Amino acids which are included in the same group can mutually be substituted.

A group: leucine, isoleucine, norleucine, valine, norvaline, alanine, 2-aminobutanoic acid, methionine, O-methylserine, t-butylglycine, t-butylalanine and cyclohexylalanine

B group: aspartic acid, glutamic acid, isoaspartic acid, isoglutamic acid, 2-aminoadipic acid and 2-aminosuberic acid

C group: asparagine and glutamine

D group: lysine, arginine, ornithine, 2,4-diaminobutanoic acid and 2,3-diaminopropionic acid

E group: proline, 3-hydroxyproline and 4-hydroxyproline

F group: serine, threonine and homoserine

G group: phenylalanine, tyrosine

For a polypeptide having an amino acid sequence in which one or more amino acid residues are deleted, substituted or added in NDH polypeptide A or known NDH polypeptides to have the energy non-production NADH dehydrogenase activity, it is advisable that the peptide has homology to a polypeptide before deletion, substitution or addition by at least 60%, usually at least 80%, especially at least 95%.

The homology of amino acid sequences or nucleotide sequences can be determined using algorism BLAST by Karlin and Altschul [Pro. Natl. Acad. Sci. USA, 90, 5873 (1993)] or FASTA [Methods Enzymol., 183, 63 (1990)]. On the basis of this algorism BLAST, a program called BLASTN or BLASTX has been developed [J. Mol. Biol., 215, 403 (1990)].

When nucleotide sequences are analyzed with BLASTN based on BLAST, parameters are, for example, score=100 and wordlength=12. When amino acid sequences are analyzed with BLASTX based on BLAST, parameters are, for example, score=50 and wordlength=3. When BLAST and Gapped BLAST programs are used, a default parameter of each program is used. Specific methods of these analyses are known (http://www.ncbi.nlm.nih.gov.).

The energy non-production NADH dehydrogenase activity of the NDH polypeptide can be measured by measuring a decrease in absorbance at 275 nm or 340 nm in a reaction solution containing energy non-production NADH dehydrogenase, ubiquinone-1 and NADH according to, for example, the description in FEMS Microbiology Letters, 204, 271 (2001).

A DNA coding for the NDH polypeptide may be any DNA coding for a polypeptide having the energy non-production NADH dehydrogenase activity.

Examples of the DNA coding for the NDH polypeptide include a DNA coding for NDH polypeptide A or coding for Corynebacterium glutamicum-derived NADH dehydrogenase possessed by plasmid pCS-CGndh carried by Escherichia coli DH5α/pCS-CGndh (FERM BP-08633) and DNAs coding for known NDH polypeptides, such as DNAs cording for polypeptides having amino acid sequences represented by SEQ ID NOs: 4, 6, 8, 10, 12, 14 and 16 and having nucleotide sequences represented by SEQ ID NOs: 3, 5, 7, 9, 11, 13 and 15.

The DNA coding for the NDH polypeptide which is used in the present invention may be a DNA which hybridizes, under stringent conditions, with a DNA having a nucleotide sequence complementary to the nucleotide sequence of NDH polypeptide A or known NDH polypeptides and which encodes a polypeptide having the energy non-production NADH dehydrogenase activity.

The DNA which hybridizes, under stringent conditions, with a DNA having a nucleotide sequence complementary to the nucleotide sequence of NDH polypeptide A or known NDH polypeptides means a DNA which is obtainable by a colony hybridization method, a plaque hybridization method, a southern blot hybridization method or the like using as a probe a part or the whole of the DNA having a nucleotide sequence complementary to a nucleotide sequence of a DNA coding for NDH polypeptide A or known NDH polypeptides.

Specifically, a DNA can be mentioned which can be identified by performing hybridization at 65° C. in the presence of from 0.7 to 1.0 mol/l of sodium chloride using a filter having fixed thereon a colony-derived or plaque-derived DNA and then washing the filter at 65° C. using an SSC solution (the SSC solution at a 1-fold concentration comprises 150 mmol/l sodium chloride and 15 mmol/l sodium citrate) at a 0.1- to 2-fold concentration.

Hybridization can be performed by a method described in Molecular Cloning, 3rd ed., Current Protocols in Molecular Biology, DNA Cloning 1: Core Techniques, A Practical Approach, Second Edition, Oxford University (1995) or the like.

Examples of the DNA which hybridizes, under stringent conditions, with the DNA having the nucleotide sequence complementary to the nucleotide sequence of the DNA coding for NDH polypeptide A or known NDH polypeptides include a DNA having homology by 75% or more, preferably 80% or more, further preferably 95% or more, to the nucleotide sequence of the DNA coding for NDH polypeptide A or known NDH polypeptides when conducting calculation using the foregoing BLAST or FASTA.

The DNA coding for the NDH polypeptide can be prepared in the following manner from microorganisms which are aerobic bacteria having energy non-production NADH dehydrogenase in electron transport systems. Examples of microorganisms having energy non-production NADH dehydrogenase in electron transport systems include so-called coryneform bacteria which are microorganisms belonging to the genus Corynebacterium (for example, Corynebacterium glutamicum), Brevibacterium, Arthrobacter, Aureobacterium, Cellulomonas, Clavibacter, Curtobacterium, Microbacterium and Pimerobacter, microorganisms belonging to the genus Escherichia (for example, Escherichia coli), microorganisms belonging to the genus Pseudomonas (for example, Pseudomonas fluorescens), microorganisms belonging to the genus Azotobacter (for example, Azotobacter vinelandii), microorganisms belonging to the genus Salmonella (for example, Salmonella typhimurium), microorganisms belonging to the genus Lactobacillus (for example, Lactobacillus plantarum), and the like.

The above microorganisms are cultured by the known method [for example, the method described in Mol. Microbiol., 20, 833 (1996)]. After the culturing, a chromosomal DNA of the microorganisms is isolated and purified by the known method (for example, the method described in Current Protocols in Molecular Biology).

The desired DNA can be obtained by the PCR method [PCR Protocols, Academic Press (1990)] using a DNA synthesized on the basis of the nucleotide sequence of the DNA coding for known NDH polypeptides as a primer and the chromosomal DNA of the microorganisms isolated and purified as a template.

Examples of the primer include DNAs having nucleotide sequences represented by SEQ ID NOs: 1 and 2, which are designed on the basis of the nucleotide sequence, represented by SEQ ID NO: 3, of ndh of a microorganism belonging to Corynebacterium glutamicum.

The desired DNA can also be prepared by the following method.

A DNA library is produced with the chromosomal DNA isolated and purified according to the method described in Molecular Cloning, 3rd ed., Current Protocols in Molecular Biology, DNA cloning 1: Core Techniques, A Practical Approach, Second Edition, Oxford University Press (1955) and the like.

As a cloning vector for producing the DNA library, a phage vector, a plasmid vector and the like can be used so long as they are autonomously replicable in Escherichia coli K12 strain. Specific examples thereof include ZAP Express [manufactured by Stratagene, Strategies, 5, 58 (1992)], λzap II (manufactured by Stratagene), λgt10, λgt11 [DNA Cloning, A Practical Approach, 1, 49 (1985)], λ TriplEx (manufactured by Clontech), λExCell (manufactured by Amersham Pharmacia Biotech), pBluescript II KS(−), pBluescript II SK(+) [manufactured by Stratagene, Nucleic Acids Research, 17, 9494 (1989)], pUC18 [Gene, 33, 103 (1985)], and the like.

The vector having a DNA incorporated therein is introduced into microorganisms belonging to Escherichia coli.

As the microorganisms belonging to Escherichia coli, any microorganisms belonging to Escherichia coli can be used. Specific examples thereof include Escherichia coli XL1-Blue MRF′ [manufactured by Stratagene, Strategies, 5, 81 (1992)], Escherichia coli C600 [Genetics, 39, 440 (1954)], Escherichia coli Y1088 [Science, 222, 778 (1983)], Escherichia coli Y1090 [Science, 222, 778 (1983)], Escherichia coli NM522 [J. Mol. Biol., 166, 1 (1983)], Escherichia coli K802 [J. Mol. Biol., 16, 118 (1966)], Escherichia coli JM109 [Gene, 38, 275 (1985)], Escherichia coli DH5α[J. Mol. Biol., 166, 557 (1983)], and the like.

A desired clone can be obtained from the resulting DNA library by a colony hybridization method, a plaque hybridization method, a southern hybridization method or the like described in an experimentation document such as Molecular Cloning, 3rd ed., Current Protocols in Molecular Biology, DNA cloning 1: Core Techniques, A practical Approach, Second Edition, Oxford University (1995).

Examples of a DNA probe used in the hybridization include a DNA having a nucleotide sequence complementary to a nucleotide sequence of a DNA coding for known NDH polypeptides or its part, a DNA synthesized on the basis of the nucleotide sequence of the DNA coding for known NDH polypeptides, a DNA fragment obtained by PCR or the like with a DNA primer designed using a known nucleotide sequence, and the like.

For example, a DNA fragment can be mentioned which is obtained from a microorganism belonging to Corynebacterium glutamicum using as a primer a DNA having nucleotide sequences represented by SEQ ID NO: 1 or 2 designed on the basis of the nucleotide sequence, represented by SEQ ID NO: 3, of ndh of the microorganism belonging to Corynebacterium glutamicum, the DNA being chemically synthesized with 8905 type DNA synthesizer manufactured by Perceptive Biosystems or the like.

The obtained DNA is introduced into a vector either as such or after cleaved with an appropriate restriction endonuclease or the like, and a nucleotide sequence of the DNA is determined by an ordinary nucleotide sequence analytical method such as a dideoxy method [Proc. Natl. Acad. Sci. USA, 74, 5463 (1977)] using ABI377DNA Sequencer (manufactured by Perkin Elmer) or the like.

Further, a primer is prepared on the basis of the determined nucleotide sequence, and the desired DNA can be obtained by the PCR method [PCR Protocols, Academic Press (1990)] using the primer and the chromosomal DNA isolated and purified as a template.

The desired DNA can also be prepared by chemical synthesis with 8905 type DNA synthesizer manufactured by Perceptive Biosystems or the like on the basis of the determined nucleotide sequence of the DNA.

As the above-obtained DNA coding for the polypeptide as used in the present invention, for example, a DNA coding for energy non-production NADH dehydrogenase possessed by plasmid pCS-CGndh carried by Escherichia coli DH5α/pCS-CGndh (FERM BP-08633) can be mentioned. This DNA is a DNA coding for NDH polypeptide A.

The microorganism used in the process for producing the amino acid in the present invention can be produced by introducing the DNA coding for the polypeptide as used in the present invention into the host microorganism.

As the method for introducing the DNA coding for the polypeptide as used in the present invention into the host microorganism, a method in which the DNA is inserted downstream of a promoter of an appropriate expression vector to form a recombinant DNA and the recombinant DNA is introduced into the host microorganism can be mentioned.

The host microorganism is not particularly limited so long as it is an aerobic bacterium. The electron transport system of the microorganism is not necessarily a system using energy non-production NADH dehydrogenase.

Examples of the host microorganism include microorganisms belonging to the genus Escherichia, Corynebacterium, Brevibacterium, Arthrobacter, Aureobacterium, Cellulomonas, Clavibacter, Curtobacterium, Microbacterium, Pimerobacter, Enterobacter, Klebsiella, Serratia, Erwinia, Bacillus, Pseudomonas, Agrobacterium, Anabaena, Chromatium, Rhodobacter, Rhodopseudomonas, Rhodospirillum, Streptomyces, Zymomonas or the like.

Examples of the microorganisms belonging to the genus Escherichia include Escherichia coli XL1-Blue, Escherichia coli XL2-Blue, Escherichia coli DH1, Escherichia coli DH5α, Escherichia coli MC1000, Escherichia coli KY3276, Escherichia coli W1485, Escherichia coli JM109, Escherichia coli HB101, Escherichia coli No. 49, Escherichia coli W3110, Escherichia coli NY49, Escherichia coli MP347 and Escherichia coli NM522.

Examples of the microorganisms belonging to the genus Corynebacterium include microorganisms belonging to Corynebacterium glutamicum (for example, Corynebacterium glutamicum ATCC 13032 and Corynebacterium glutamicum ATCC 13869), Corynebacterium ammoniagenes (for example, Corynebacterium ammoniagenes ATCC 6872 and Corynebacterium ammoniagenes ATCC 21170), Corynebacterium acetoacidophilum (for example, Corynebacterium acetoacidophilum ATCC 13870), and the like.

Examples of the microorganisms belonging to the genus Brevibacterium include microorganisms belonging to Brevibacterium immariophilum, Brevibacterium saccharolyticum, Brevibacterium flavum, Brevibacterium lactofermentum, and the like.

Examples of the microorganisms belonging to the genus Arthrobacter include microorganisms belonging to Arthrobacter citreus, Arthrobacter globiformis, and the like.

Examples of the microorganisms belonging to the genus Aureobacterium include microorganisms belonging to Aureobacterium flavescens, Aureobacter iumsaperdae, Aureobacterium testaceum, and the like.

Examples of the microorganisms belonging to the genus Cellulomonas include microorganisms belonging to Cellulomonas flavigena, Cellulomonas carta, and the like.

Examples of the microorganisms belonging to the genus Clavibacter include microorganisms belonging to Clavibacter michiganensis, Clavibacter rathayi, and the like.

Examples of the microorganisms belonging to the genus Curtobacterium include microorganisms belonging to Curtobacterium albidum, Curtobacter iumcitreum, Curtobacerium luteum, and the like.

Examples of the microorganisms belonging to the genus Microbacterium include microorganisms belonging to Microbacterium ammoniaphilum (for example, Microbacterium ammoniaphilum ATCC 15354), Microbacterium lacticum, Microbacterium imperiale, and the like.

Examples of the microorganisms belonging to the genus Pimerobacter include microorganisms belonging to Pimerobacter simplex, and the like.

Examples of the microorganisms belonging to the genus Enterobacter include microorganisms belonging to Enterobacter agglomerans (for example, Enterobacter agglomerans ATCC 1228), Enterobacter aerogenes, Enterobacter amnigenus, Enterobacter asburiae, Enterobacter cloacae, Enterobacter dissolvens, Enterobacter gergoviae, Enterobacter hormaechei, Enterobacter intermedius, Enterobacter nimipressuralis, Enterobacter sakazakii, Enterobacter taylorae, and the like.

Examples of the microorganisms belonging to the genus Klebsiella include microorganisms belonging to Klebsiella planticola, and the like.

Examples of the microorganisms belonging to the genus Serratia include microorganisms belonging to Serratia ficaria, Serratia fonticola, Serratia liquefaciens, Serratia entomophila, Serratia grimessi, Serratia proteamaculans, Serratia odorifera, Serratia plymuthica, Serratia rubidaea, Serratia marcescens, and the like.

Examples of the microorganisms belonging to the genus Erwinia include microorganisms belonging to Erwinia uredovora, Erwinia carotovora, Erwinia ananas, Erwinia herbicola, Erwinia punctata, Erwinia terreus, Erwinia cacticida, Erwinia chrysanthemi, Erwinia mallotivora, Erwinia persicinus, Erwinia psidii, Erwinia quercina, Erwinia rhapontici, Erwinia rubrifaciens, Erwinia salicis, and the like.

Examples of the microorganisms belonging to the genus Bacillus include microorganisms belonging to Bacillus subtilis, Bacillus megaterium, Bacillus amyloliquefaciens, Bacillus coagulans, Bacillus licheniformis, Bacillus pumilus, and the like.

Examples of the microorganisms belonging to the genus Pseudomonas include microorganisms belonging to Pseudomonas putida, and the like.

Examples of the microorganisms belonging to the genus Agrobacterium include microorganisms belonging to Agrobacterium radiobacter, Agrobacterium rhizogenes, Agrobacterium rubi, and the like.

Examples of the microorganisms belonging to the genus Anabaena include microorganisms belonging to Anabaena cylindrica, Anabaena doliolum, Anabaena flosaquae, and the like.

Examples of the microorganisms belonging to the genus Chromatium include microorganisms belonging to Chromatium buderi, Chromatium tepidum, Chromatium vinosum, Chromatium warmingii, Chromatium fluviatile, and the like.

Examples of the microorganisms belonging to the genus Rhodobacter include microorganisms belonging to Rhodobacter capsulatus, Rhodobacter sphaeroides, and the like.

Examples of the microorganisms belonging to the genus Rhodopseudomonas include microorganisms belonging to Rhodopseudomonas blastica, Rhodopseudomonas marina, Rhodopseudomonas palustris, and the like.

Examples of the microorganisms belonging to the genus Rhodospirillum include Rhodospirillum rubrum, Rhodospirillum salexigens, Rhodospirillum salinarum, and the like.

Examples of the microorganisms belonging to the genus Streptomyces include microorganisms belonging to Streptomyces ambofaciens, Streptomyces aureofaciens, Streptomyces aureus, Streptomyces fungicidicus, Streptomyces griseochromogenes, Streptomyces griseus, Streptomyces lividans, Streptomyces olivogriseus, Streptomyces rameus, Streptomyces tanashiensis, Streptomyces vinaceus, and the like.

Examples of the microorganisms belonging to the genus Zymomonas include microorganisms belonging to Zymomonas mobilis, and the like.

Among the foregoing host microorganisms, the microorganisms belonging to the genus Corynebacterium, Brevibacterium, Arthrobacter, Aureobacterium, Cellulomonas, Clavibacter, Curtobacterium, Microbacterium, Pimerobacter, Escherichia or Bacillus are preferably used. The microorganisms belonging to the genus Corynebacterium or Escherichia are more preferably used. The microorganisms belonging to the genus Corynebacterium are further preferably used.

As the method for introducing a recombinant DNA into a host microorganism, any method capable of introducing a DNA into the host microorganism is available. Examples thereof include a method using a calcium ion [Proc. Natl. Acad. Sci., USA, 69, 2110 (1972)], a protoplast method (Japanese Published Unexamined Patent Application No. 248394/1988), an electroporation method (Nucleic Acids, Res., 16, 6127 (1988)], and the like.

As the expression vector, a vector capable of autonomous replication or incorporation into a chromosome in a host microorganism and containing a promoter in a site where the DNA coding for the polypeptide as used in the present invention can be transcribed is used.

Examples thereof include pBTrp2, pBTac1, pBTac2 (all manufactured by Boehringer Mannheim), pHelix1 (manufactured by Roche Diagnostics), pKK233-2 (manufactured by Amersham Pharmacia Biotech), pSE280 (manufactured by Invitrogen), pGEMEX-1 (manufactured by Promega), pQE-8 (manufactured by Qiagen), pET-3 (manufactured by Novagen), pKYP10 (Japanese Published Unexamined Patent Application No. 110600/1983), pKYP200 [Agric. Biol. Chem., 48, 669 (1984)], pLSA1 [Agric. Biol. Chem., 53, 277 (1989)], pGEL1 [Proc. Natl. Acad. Sci., USA, 82, 4306 (1985)], pBluescript II SK (+), pBluescript II KS(−) (manufactured by Stratagene), pTrS30 [prepared from Escherichia coli JM109/pTrS30 (FERM BP-5407)], pTrS32 [prepared from Escherichia coli JM109/pTrS32 (FERM BP-5408)], pPAC31 (WO 98/12343), pUC19 [Gene, 33, 103 (1985)], pSTV28 (manufactured by Takara Shuzo), pUC118 (manufactured by Takara Shuzo), pPA1 (Japanese Published Unexamined Patent Application No. 233798/1988), pCG116, pCG1 (Japanese Published Unexamined Patent Application No. 277082/1994), pCS299P (WO 00/63388) and the like.

As the promoter, any promoter functioning in the host microorganisms may be used. Examples thereof include promoters derived from microorganisms belonging to Escherichia coli, phages or the like, such as trp promoter (Pₜᵣₚ) lac promoter (P_{lac}), P_{L} promoter, P_{R} promoter and P_{SE} promoter, SPO1 promoter, SPO2 promoter, penP Promoter and the like. Further, artificially modified promoters, such as a promoter in which two Pₜᵣₚs are arranged in series, tac promoter, lacT7 promoter and let I promoter.

When the host microorganism is a microorganism belonging to the genus Corynebacterium, P54-6 promoter [Appl. Microbiol. Biotechnol., 53, 674-679 (2000)] is also mentioned. When the host microorganism is a microorganism belonging to the genus Bacillus, xylA promoter [Appl. Microbiol. Biotechnol., 35, 549-599 (1991)] is also mentioned.

It is preferred that the recombinant DNA is capable of autonomous replication in host microorganisms, and at the same time, is a recombinant DNA comprising the foregoing promoter, a ribosome binding sequence, a DNA coding for the polypeptide as used in the present invention and a transcription termination sequence. A promoter-controlling gene may be incorporated therein.

It is preferred that there is an appropriate distance (for example, from 6 to 18 nucleotides) between the Shine-Dalgarno sequence as a ribosome binding sequence and an initiation codon.

Although the transcription termination sequence is not necessarily required, it is preferred that the transcription termination sequence lies immediately downstream of the structural gene.

As the recombinant DNA, for example, plasmid pCS-CGndh carried by Escherichia coli DH5α/pCS-CGndh (FERM BP-08633) can be mentioned.

Examples of the microorganisms of the present invention obtained by the foregoing method include Corynebacterium glutamicum LS-22/pCS-CGndh, Corynebacterium glutamicum ATCC 14752/pCS-CGndh and Corynebacterium glutamicum FERM BP-1069/pCS-CGndh.

The DNA coding for the polypeptide, as used in the present invention, which is introduced into the host microorganism may be present in a recombinant DNA in the microorganism or may be incorporated in a chromosome.

As the method in which the DNA is incorporated into the chromosome, for example, a method using a phage or a transposon described in Escherichia coli and Salmonella typhimurium, 1996, p. 2325, p. 2339, American Society for Microbiology or the like.

An amino acid can be produced by culturing in a medium a microorganism obtained by introducing the DNA coding for the polypeptide as used in the present invention (hereinafter abbreviated as a microorganism of the present invention), forming and accumulating the amino acid in the culture and collecting the amino acid from the culture.

The amino acid is not particularly limited, and examples thereof include amino acids biosynthesized from 2-oxoglutaric acid, such as L-glutamic acid and L-glutamine, amino acids biosynthesized from oxaloacetic acid, such as L-aspartic acid and L-asparagine, amino acids biosynthesized from aspartic acid, such as L-lysine, L-methionine and L-threonine, amino acids biosynthesized from L-glutamic acid, such as L-arginine, L-proline and L-citrulline, amino acids biosynthesized from pyruvic acid, such as L-valine, L-leucine and L-isoleucine, amino acids biosynthesized from 3-phosphoglyceric acid, such as L-serine, L-cysteine and glycine, amino acids biosynthesized from chorismic acid, such as L-tryptophan, L-tyrosine and L-phenylalanine, L-histidine, and the like.

The medium used in culturing may be either a natural medium or a synthetic medium so long as it contains a carbon source, a nitrogen source, inorganic salts and the like which can be assimilated by the microorganisms of the present invention, in which the microorganisms can be grown and a desired amino acid is produced efficiently.

Any carbon source capable of being assimilated by the microorganisms of the present invention may be used. Glucose, fructose, sucrose, molasses containing the same, carbohydrates such as starch and starch hydrolyzate, organic acids such as acetic acid and propionic acid, alcohols such as methanol, ethanol and propanol, and the like can be used.

As the nitrogen source, ammonia., inorganic or organic acid ammonium salts such as ammonium chloride, ammonium sulfate, ammonium acetate and ammonium phosphate, other nitrogen-containing compounds, peptone, meat extract, yeast extract, corn steep liquor, casein hydrolyzate, soybean cake hydrolyzate, various fermented bacteria, digested substances thereof, and the like can be used.

As the inorganic salt, potassium dihydrogenphosphate, dipotassium hydrogenphosphate, magnesium phosphate, magnesium sulfate, sodium chloride, ferrous sulfate, manganese sulfate, copper sulfate, calcium carbonate and the like can be used.

The culturing is conducted under aerobic conditions of shake culturing, or submerged culturing with stirring or the like. The culturing temperature is preferably from 15° C. to 50° C., more preferably from 20° C. to 45° C. The culturing time is usually from 5 hours to 7 days, preferably from 12 hours to 4 days. During the culturing, pH is maintained at from 3 to 9 as required. The pH is adjusted with an inorganic or organic acid, an alkaline solution, urea, calcium carbonate, ammonia or the like.

During the culturing, an antibiotic such as penicillin, ampicillin or tetracycline may be added to the medium as required.

When culturing a microorganism into which a DNA is introduced with a recombinant DNA using a inducible promoter as the promoter, an inducer may be added to a medium as required. For example, when culturing a microorganism into which a DNA is introduced with a recombinant DNA using lac promoter, isopropyl-β-D-thiogalactopyranoside or the like may be added to a medium, and when culturing a microorganism into which a DNA is introduced with a recombinant DNA using trp promoter, indole acrylic acid or the like may be added to a medium.

An amino acid can be recovered from a culture after completion of the culturing by methods usually employed for isolation of an amino acid, such as a method using activated carbon, a method using an ion exchange resin, a crystallization method and a precipitation method which are employed either singly or in combination.

Examples are described below. However, the present invention is not limited to the following Examples.

### EXAMPLE 1

(1) Corynebacterium glutamicum LS-22 strain was inoculated in LB medium [medium containing 10 g/L bactotrypton (manufactured by Difco), 5 g/L yeast extract (manufactured by Difco) and 5 g/L sodium chloride], and was cultured overnight at 30° C. After the culturing, a chromosomal DNA of the microorganism was isolated and purified according to the method of Eikmanns et al [Microbiol., 140, 1817 (1994)].

A DNA having nucleotide sequence represented by SEQ ID NO: 1 or 2 was synthesized on the basis of a nucleotide sequence of ndh of Corynebacterium glutamicum ATCC 13032 represented by SEQ ID NO: 3.

PCR was performed in 40 μL of a reaction solution containing 2.5 units of PfuDNA polymerase (manufactured by Stratagene) and dNTPs (dATP, dGTP, dCTP and dTTP) in amounts of 200 μmol/L each using the above chromosomal DNA (0.1 μg) as a template.

After completion of the reaction, 4 μL of the resulting reaction solution was subjected to agarose gel electrophoresis to confirm that 1.9 kb of a fragment corresponding to ndh of known Corynebacterium glutamicum was amplified. Then, the remaining reaction solution and an equal amount of a TE [solution containing 10 mmol/L Tris-hydrochloride (pH 8.0) and 1 mmol/L ethylenediaminetetraacetic acid] saturated phenol/chloroform (1 vol/1 vol) solution were mixed. A 2-fold amount of cold ethanol was added to an upper layer obtained by centrifugal separation of the solution, and the mixture was allowed to stand at −80° C. for 30 minutes. The solution was centrifuged to obtain a DNA, and the DNA was dissolved in 20 μL of TE.

5 μL of the DNA-containing solution and 0.06 μg of pGEM^{R}-T Easy vector (manufactured by Promega) were reacted at 16° C. for 16 hours using a ligation kit, pGEM^{R}-T Easy vector system (manufactured by Promega) to ligate a ndh-containing DNA fragment with pGEM^{R}-T Easy vector.

Escherichia coli DH5α strain was transformed by an electroporation method [Nucleic acid Res., 16, 6127-6145 (1988)] using the reaction solution after the ligation reaction.

The resulting transformant was spreaded on an LB agar medium containing 100 μg/mL ampicillin, and cultured overnight at 30° C. A plasmid was extracted from colonies grown in the agar medium in a usual manner, and its structure was analyzed with a restriction endonuclease to confirm that the plasmid was a plasmid in which the DNA fragment containing ndh of Corynebacterium glutamicum was inserted in pGEM^{R}-T Easy vector. This plasmid was designated pT-CGndh.

1 μg of plasmid pT-CGndh was cleaved with restriction endonucleases KpnI and SalI, and a solution containing the resulting DNA fragment was subjected to agarose gel electrophoresis to separate the DNA fragment of approximately 2 kb.

0.2 μg of pCS299P (WO 00/63388) as an expression vector for coryneform bacteria was cleaved with restriction endonucleases KpnI and SalI, and a solution containing the resulting DNA fragment was subjected to agarose gel electrophoresis to separate the DNA fragment of approximately 5.4 kb.

The above-obtained ndh-containing DNA fragment of approximately 2 kb and the cleaved fragment of pCS299P (approximately 5.4 kb) were reacted at 16° C. for 16 hours using a ligation kit (manufactured by Takara Shuzo) for ligation.

Corynebacterium glutamicum LS-22 strain was transformed with the reaction solution after the ligation reaction by an electroporation method [Nucleic acid Res., 16, 6127-6145 (1988)].

The resulting transformant was spreaded on an LB agar medium containing 25 μg/mL of kanamycin, and cultured at 30° C. for 1 day. A plasmid was extracted from colonies grown in the agar medium in a usual manner, and its structure was analyzed with a restriction endonuclease to confirm that the plasmid was a plasmid in which the ndh-containing DNA fragment was inserted in pCS299P. This plasmid was designated pCS-CGndh.

Escherichia coli DH5α/pCS-CGndh containing plasmid pCS-CGndh was deposited as FERM BP-08633 on Feb. 19, 2004 in International Patent Organism Depositary, National Institute of Advanced Industrial Science and Technology, AIST Tsukuba Central 6, 1-1-1 Higashi, Tsukuba Ibaraki, 305-8566, Japan.

(2) Each of Corynebacterium glutamicum LS-22 strain and Corynebacterium glutamicum LS-22/pCS-CGdh strain obtained by introducing pCS-CGndh into Corynebacterium glutamicum LS-22 strain was inoculated into 5 mL of LB medium in a test tube, and cultured overnight at 30° C. while being shaken.

One mL of the culture was added to a 500-mL conical flask containing 100 mL of MG medium [medium containing 10 g/L glucose, 3 g/L potassium dihydrogenphosphate, 3 g/L dipotassium hydrogenphosphate, 2 g/L ammonium chloride, 2 g/L urea, 0.5 g/L magnesium sulfate 7-hydrate, 10 mg/L iron sulfate 10-hydrage, 1 mg/L manganese sulfate 7-hydrate, 30 mg/L biotin, 1 mg/L thiamine hydrochloride, 20 mg/L cysteine hydrochloride, 0.5 g/L casamino acid and 1 mL/L metal mix (solution containing 990 mg/L iron sulfate 7-hydrate, 880 mg/L zinc sulfate 7-hydrate, 393 mg/L copper sulfate 5-hydrate, 72 mg/L manganese chloride 4-hydrate, 88 mg/L sodium tetraborate 10-hydrate and 37 mg/L ammonium paramolybdate 4-hydrate)], and was cultured at 30° C. and 220 rpm for 80 hours while being shaken. After the culturing, a concentration of glutamic acid in a culture supernatant was measured by HPLC.

In the HPLC analysis, the culture supernatant was subjected to column AQ-312 (manufactured by YMC) (mobile phase: solution of pH 2.4 containing 2.94 g/L sodium citrate, 1.42 g/L sodium sulfate, 17 mL/L n-propanol and 3 g/L sodium laurylsulfate), and was mixed with a reaction solution (solution containing 18.5 g/L boric acid, 11 g/L NaOH, 0.6 g/L orthophthalaldehyde, 2 ml/L mercaptoethanol and 3 mL/L Brige-35). The mixture was subjected to fluorescence analysis with an excitation wavelength of 345 nm and an absorption wavelength of 455 nm.

As the result, Corynebacterium glutamicum LS-22 strain accumulated 1.5 g/L glutamic acid in the culture, while Corynebacterium glutamicum LS-22/pCS-CGndh strain accumulated 2.3 g/L glutamic acid.

### EXAMPLE 2

pCS-CGndh was introduced into Corynebacterium glutamicum ATCC 14752 strain as in Example 1 using the electroporation method to obtain Corynebacterium glutamicum ATCC 14752/pCS-CGndh strain.

Each of Corynebacterium glutamicum ATCC 14752 strain and Corynebacterium glutamicum ATCC 14752/pCS-CGndh strain was inoculated in a test tube containing 5 mL of GS medium [medium of pH 7.2 containing 70 g/L glucose, 10 g/L corn steep liquor, 10 g/L meat extract, 10 g/L yeast extract, 5 g/L ammonium sulfate, 0.5 g/L potassium dihydrogenphosphate, 1.5 g/L dipotassium hydrogenphosphate, 0.5 g/L magnesium sulfate 7-hydrate, 10 mg/L iron sulfate 10-hydrate, 10 mg/L manganese sulfate 7-hydrate, 0.8 mg/L copper sulfate 5-hydrate, 8.3 g/L urea, 5 μg/L biotin and 1 mg/L thiamine hydrochloride), and was cultured at 30° C. for 24 hours while being shaken.

2.5 mL of this culture was added to a conical flask containing 25 ml of GP medium [medium of pH 7.2 containing 116 g/L glucose, 4 g/L fructose, 50 g/L ammonium chloride, 10 mg/L nicotinic acid, 0.7 g/L potassium dihydrogenphosphate, 0.7 g/L dipotassium hydrogenphosphate, 0.5 g/L magnesium sulfate 7-hydrate, 20 mg/L iron sulfate 10-hydrate, 20 mg/L manganese sulfate 7-hydrate, 0.8 mg/L copper sulfate 5-hydrate, 5 g/L urea, 0.5 μg/L biotin, 1 mg/L thiamine hydrochloride and 50 g/L calcium carbonate), and was cultured at 30° C. and 220 rpm for 72 hours while being shaken.

After the culturing, the amount of glutamine accumulated in the culture supernatant was measured under the HPCL conditions described in Example 1.

Consequently, the amount of glutamine accumulated in Corynebacterium glutamicum ATCC 14752 strain was 32.2 g/L, while the amount of glutamine accumulated in Corynebacterium glutamicum ATCC 14752/pCS-CGndh strain was 33.3 g/L.

### EXAMPLE 3

pCS-CGndh was introduced into Corynebacterium glutamicum FERM BP-1069 strain as in Example 1 using the electroporation method to obtain Corynebacterium glutamicum FERM BP-1069/pCS-CGndh strain.

Each of Corynebacterium glutamicum FERM BP-1069 strain and Corynebacterium glutamicum FERM BP-1069/pCS-CGndh strain was inoculated in a test tube containing 5 mL of LS medium [medium of pH 7.2 containing 50 g/L sucrose, 30 g/L corn steep liquor, 20 g/L meat extract, 20 g/L casamino acid, 8 g/L ammonium sulfate, 2 g/L potassium dihydrogenphosphate, 0.5 g/L magnesium sulfate 7-hydrate, 3 g/L urea, 20 g/L peptone, 10 mg/L iron sulfate 10-hydrate, 10 mg/L zinc sulfate 7-hydrate, 20 mg/L nicotinic acid, 10 mg/L calcium pantothenate, 0.1 mg/L biotin, 1 mg/L thiamine hydrochloride and 10 g/L calcium carbonate], and was cultured at 30° C. for 24 hours while being shaken.

0.5 mL of this culture was added to a test tube containing 5 mL of LP medium (medium of pH 7.0 containing 100 g/L molasses (as a sugar content), 45 g/L ammonium sulfate, 3 g/L urea, 0.5 g/L potassium dihydrogenphosphate, 0.5 g/L magnesium sulfate 7-hydrate, 0.3 mg/L biotin and 30 g/L calcium carbonate], and was cultured at 30° C. and 220 rpm for 72 hours while being shaken. After completion of the culturing, a concentration of lysine in a culture supernatant was measured by HPLC.

In the HPLC analysis, the culture supernatant was subjected to column ODS-80TS (manufactured by TOSOH) (mobile phase: solution of pH 6.0 containing 2.94 g/L sodium citrate, 1.42 g/L sodium sulfate, 300 mL/L acetonitrile and 3 g/L sodium laurylsulfate), and was mixed with a reaction solution (solution containing 18.5 g/L boric acid, 11 g/L sodium hydroxide, 0.6 g/L orthophthalaldehyde, 2 ml/L mercaptoethanol and 3 mL/L Brige-35). The mixture was subjected to fluorescence analysis with an excitation wavelength of 345 nm and an absorption wavelength of 455 nm.

As a result, the amount of lysine accumulated in Corynebacterium glutamicum FERM BP-1069 strain was 24.8 g/L, while the amount of lysine accumulated in Corynebacterium glutamicum FERM BP-1069/pCS-CGndh strain was 28.6 g/L.

### INDUSTRIAL APPLICABILITY

According to the present invention, an industrially advantageous process for producing an amino acid can be provided.

Sequence Listing Free Text

SEQ ID NO: 1—description of an artificial sequence: synthetic DNA

SEQ ID NO: 2—description of an artificial sequence: synthetic DNA

1. A process for producing an amino acid, which comprises culturing, in a medium, a microorganism obtainable by introducing a DNA coding for energy non-production NADH dehydrogenase, forming and accumulating an amino acid in a culture, and recovering the amino acid from the culture.

2. The process according to claim 1, wherein the DNA coding for energy non-production NADH dehydrogenase is a DNA derived from a microorganism selected from the group consisting of microorganisms belonging to the genus Corynebacterium, Escherichia, Pseudomonas, Azotobacter, Salmonella or Lactobacillus, or a DNA which hybridizes, under stringent conditions, with a DNA having a nucleotide sequence complementary to the nucleotide sequence of the DNA.

3. The process according to claim 1, wherein the DNA coding for energy non-production NADH dehydrogenase is a DNA derived from a microorganism selected from the group consisting of microorganisms belonging to the species Corynebacterium glutamicum, Corynebacterium diphtheriae, Escherichia coli Pseudomonas fluorescens, Azotobacter vinelandii, Salmonella typhimurium or Lactobacillus plantarum, or a DNA which hybridizes, under stringent conditions, with a DNA having a nucleotide sequence complementary to the nucleotide sequence of the DNA.

4. The process according to claim 1, wherein the DNA coding for energy non-production NADH dehydrogenase is a DNA having a nucleotide sequence selected from the group consisting of nucleotide sequences represented by SEQ ID NOs: 3, 5, 7, 9, 11, 13 and 15, or a DNA which hybridizes, under stringent conditions, with a DNA having a nucleotide sequence complementary to the nucleotide sequence.

5. The process according to claim 1, wherein the DNA coding for energy non-production NADH dehydrogenase is a DNA coding for energy non-production NADH dehydrogenase possessed by a plasmid pCS-CGndh carried by Escherichia coli DH5α/pCS-CGndh (FERM BP-08633) or a DNA which hybridizes, under stringent conditions, with a DNA having a nucleotide sequence complementary to the nucleotide sequence of the DNA and which encodes a polypeptide having the energy non-production NADH dehydrogenase activity.

6. The process according to claim 1, wherein the energy non-production NADH dehydrogenase is a polypeptide having an amino acid sequence selected from the group consisting of amino acids sequences represented by SEQ ID NOs: 4, 6, 8, 10, 12, 14 and 16, or a polypeptide comprising an amino acid sequence wherein one or more amino acid residues are deleted, substituted or added in the amino acid sequence of the polypeptide and having the energy non-production NADH dehydrogenase activity.

7. The process according to claim 1, wherein the energy non-production NADH dehydrogenase is a polypeptide encoded by the DNA coding for energy non-production NADH dehydrogenase possessed by a plasmid pCS-CGndh carried by Escherichia coli DH5α/pCS-CGndh (FERM BP-08633) or a polypeptide comprising an amino acid sequence wherein one or more amino acid residues are deleted, substituted or added in the amino acid sequence of the polypeptide and having the energy non-production NADH dehydrogenase activity.

8. The process according to claim 2, wherein the microorganism into which the DNA coding for energy non-production NADH dehydrogenase is introduced is a microorganism selected from the group consisting of microorganisms belonging to the genus Escherichia, Corynebacterium, Brevibacterium, Arthrobacter, Aureobacterium, Cellulomonas, Clavibacter, Curtobacterium, Microbacterium, Pimerobacter or Bacillus.

9. The process according to claim 2, wherein the microorganism into which the DNA coding for energy non-production NADH dehydrogenase is introduced is a microorganism belonging to the genus Escherichia.

10. The process according to claim 2, wherein the microorganism into which the DNA coding for energy non-production NADH dehydrogenase is introduced is a microorganism belonging to the species Escherichia coli.

11. The process according to claim 2, wherein the microorganism into which the DNA coding for energy non-production NADH dehydrogenase is introduced is a microorganism belonging to the genus Corynebacterium.

12. The process according to claim 2, wherein the microorganism into which the DNA coding for energy non-production NADH dehydrogenase is introduced is a microorganism selected from the group consisting of microorganisms belonging to the species Corynebacterium glutamicum, Corynebacterium flavum, Corynebacterium lactofermentum, or Corynebacterium efficasis.

13. The process according to claim 2, wherein the microorganism into which the DNA coding for energy non-production NADH dehydrogenase is introduced is a microorganism belonging to the species Corynebacterium glutamicum.

14. The process according to claim 2, wherein the amino acid is an amino acid selected from the group consisting of L-glutamic acid, L-glutamine, L-aspartic acid, L-asparagine, L-lysine, L-methionine, L-threonine, L-arginine, L-proline, L-citrulline, L-valine, L-leucine, L-isoleucine, L-serine, L-cysteine, glycine, L-triptophan, L-thyrosine, L-phenylalanine and L-histidine.

15. The process according to claim 2, wherein the amino acid is an amino acid selected from the group consisting of L-glutamic acid, L-glutamine and L-lysine.

16. A microorganism which belongs to the genus Corynebacterium, and is obtainable by introducing a DNA coding for energy non-production NADH dehydrogenase.

17. A microorganism which belongs to the species Corynebacterium glutamicum, and is obtainable by introducing a DNA coding for energy non-production NADH dehydrogenase.

18. The microorganism according to claim 16 or 17, wherein the DNA coding for energy non-production NADH dehydrogenase is a DNA derived from a microorganism selected from the group consisting of microorganisms belonging to the genus Corynebacterium, Escherichia, Pseudomonas, Azotobacter, Salmonella or Lactobacillus, or a DNA which hybridizes, under stringent conditions, with a DNA having a nucleotide sequence complementary to the nucleotide sequence of the DNA.

19. The microorganism according to claim 16 or 17, wherein the DNA coding for energy non-production NADH dehydrogenase is a DNA derived from a microorganism selected from the group consisting of microorganisms belonging to the species Corynebacterium glutamicum, Corynebacterium diphtheriae, Escherichia coli, Pseudomonas fluorescens, Azotobacter vinelandii, Salmonella typhimurium or Lactobacillus plantarum, or a DNA which hybridizes, under stringent conditions, with a DNA having a nucleotide sequence complementary to the nucleotide sequence of the DNA.

20. The microorganism according to claim 16 or 17, wherein the DNA coding for energy non-production NADH dehydrogenase is a DNA having a nucleotide sequence selected from the group consisting of nucleotide sequences represented by SEQ ID NOs: 3, 5, 7, 9, 11, 13 and 15, or a DNA which hybridizes, under stringent conditions, with a DNA having a nucleotide sequence complementary to the nucleotide sequence.

21. The microorganism according to claim 16 or 17, wherein the DNA coding for energy non-production NADH dehydrogenase is a DNA coding for energy non-production NADH dehydrogenase possessed by a plasmid pCS-CGndh carried by Escherichia coli DH5α/pCS-CGndh (FERM BP-08633) or a DNA which hybridizes, under stringent conditions, with a DNA having a nucleotide sequence complementary to the nucleotide sequence of the DNA and which encodes a polypeptide having the energy non-production NADH dehydrogenase activity.

22. The microorganism according to claim 16 or 17, wherein the energy non-production NADH dehydrogenase is a polypeptide having an amino acid sequence selected from the group consisting of amino acids sequences represented by SEQ ID NOs: 4, 6, 8, 10, 12, 14 and 16, or a polypeptide comprising an amino acid sequence wherein one or more amino acid residues are deleted, substituted or added in the amino acid sequence of the polypeptide and having the energy non-production NADH dehydrogenase activity.

23. The microorganism according to claim 16 or 17, wherein the energy non-production NADH dehydrogenase is a polypeptide encoded by a DNA possessed by a plasmid pCS-CGndh carried by Escherichia coli DH5α/pCS-CGndh (FERM BP-08633) or a polypeptide comprising an amino acid sequence in which one or more amino acid residues are deleted, substituted or added in the amino acid sequence of the polypeptide and having the energy non-production NADH dehydrogenase activity.

24. Corynebacterium glutamicum ATCC 14752/pCS-CGndh or Corynebacterium glutamicum FERM BP-1069/pCS-CGndh.

25. Escherichia coli DH5α/pCS-CGndh (FERM BP-08633).

26. Plasmid pCS-CGndh carried by Escherichia coli DH5α/pCS-CGndh (FERM BP-08633).


SEQUENCE LISTING (16 sequences)

SEQ ID NO: 1 - DNA, 30 bp, Artificial
  Synthetic DNA
ctgcttgccctgcaggtgcaccagcaaacg

SEQ ID NO: 2 - DNA, 30 bp, Artificial
  Synthetic DNA
cgagctgcgcgacaaccaggaattcagcgg

SEQ ID NO: 3 - DNA, 1404 bp, Corynebacterium glutamicum ATCC13032
  CDS
atgtcagttaacccaacccgccccgaaggcggccgtcaccacgtcgtcgtcatcggttctggttttggtggcctttttgctgccaagaacctggccaaggcagacgtcgatgtcactctgattgaccgcaccaaccaccacctcttccagccactgctgtaccaagtggcaaccggtatcctctcctccggtgaaatcgcaccttccactcgacagatcctgggctcccaggaaaacgtcaacgtcatcaagggcgaagtcaccgacatcaacgtcgagtcccagactgtgaccgcctccctgggcgagttcacccgcgtttttgagtacgattccttggtcgttggtgctggcgcaggtcagtcctacttcggcaatgatcacttcgctgagttcgcacctggcatgaagtccatcgacgatgcactggagattcgtgcacgcatcatcggtgctttcgagcgcgctgagatctgcgaggatccagctgagcgcgaacgcctgctcaccttcgtcgttgttggcgctggcccaaccggtgttgagcttgctggccagttggctgagatggctcaccgcacccttgctggtgagtacaagaacttcaacaccaactccgcaaagatcatcctgcttgatggtgctccacaggttcttcctccattcggtaagcgcctaggccgcaacgcacagcgcaccctggaaaagatgggtgtcaacgttcgcctgaacgctatggtcaccaacgttgacgctacctcggtcacctacaagaccaaggacggcgaagagcacaccatcgaatctttctgcaagatttggtccgctggtgttgcggcatccccactgggcaagctcgtcgcagagcagaccggtgttgagaccgaccgcgcaggccgcgtcatggttaacgatgacctgtctgttggcgatcagaagaacgtcttcgttgttggcgacatgatgaactacaacaacctccctggtgttgctcaggtagcaatccagagtggtgagtacgttgctgagcagatcgaagctgaggttgaaggccgctccaacaccgagcgcgaagctttcgattacttcgacaagggctccatggctaccatttcccgcttctccgcagtggtgaagatgggcaaggttgaggtcaccggcttcatcggttgggttctgtggttggctgttcacatcatgttcctggttggcttccgcaaccgtttcgtctccgcaatcagctggggcctgaacgcactgtcccgcaagcgttggaacctggcaaccacccgccagcagctccactcacgcaccacgctgtccaagttcgctcacgagcttgaggaagcatcttctgatcttccaatcgagctgcgcgacaaccagcgtttcagcggaaagtaa

SEQ ID NO: 4 - protein, 467 aa, Corynebacterium glutamicum ATCC13032
MSVNPTRPEGGRHHVVVIGSGFGGLFAAKNLAKADVDVTLIDRTNHHLFQPLLYQVATGILSSGEIAPSTRQILGSQENVNVIKGEVTDINVESQTVTASLGEFTRVFEYDSLVVGAGAGQSYFGNDHFAEFAPGMKSIDDALEIRARIIGAFERAEICEDPAERERLLTFVVVGAGPTGVELAGQLAEMAHRTLAGEYKNFNTNSAKIILLDGAPQVLPPFGKRLGRNAQRTLEKMGVNVRLNAMVTNVDATSVTYKTKDGEEHTIESFCKIWSAGVAASPLGKLVAEQTGVETDRAGRVMVNDDLSVGDQKNVFVVGDMMNYNNLPGVAQVAIQSGEYVAEQIEAEVEGRSNTEREAFDYFDKGSMATISRFSAVVKMGKVEVTGFIGWVLWLAVHIMFLVGFRNRFVSAISWGLNALSRKRWNLATTRQQLHSRTTLSKFAHELEEASSDLPIELRDNQRFSGK

SEQ ID NO: 5 - DNA, 1362 bp, Corynebacterium diphtheriae
  CDS
atgactaacaccccatttcgcccagaaggtggacgccaccacgttgtagttattggctccggcttcggtggactattcgcagttcaaaacctcaaagatgcagatgtcgatatcaccctcatcgaccggacaaaccaccaccttttccagccgttgctttaccaagtagcaaccggtatcttgtcgtctggtgaaatcgcaccacaaacgcgtcaagttcttgcacagcaaaataatgtgcacgttcttaaggctgaagtcaccgacattgacaccgaatcgaagacggtcgtcgcagacttggatgattattctaaaacaattgaatacgattccctgatcgtcgccgctggtgcaggtcagtcttacttcggaaatgatcacttcgcggaattcgcgccgggtatgaaaacaatcgatgatgcactcgaactgcgtgcgcgcatcatcggcgctttcgaacgcgcagaaatgtgcgaagatcccaaagaacgtgaacgcctcttgacttttgttatcgttggcgcaggaccaacaggcgtagaacttgcaggtcagctggccgaaatggcacaccgcacgttgtctggagagtacacgcagttcacgccttccaacgcgaagatcatcctgcttgacggcgctcctcaggtgcttccaccgttcggcaagcgtttgggtcgtactgcacagcgtgaattagaaaagattggtgtaacggtcaagctgaacgctatcgttaccggcgtagacgaaaactcagtgacatataagtccaccgttgatgattctttgcacacgatcgattccttctgcaagatctggtcggcaggcgtagccgcttccccactaggcaaactagttgcagagcagcttggtgtcgaggttgatcgtgcaggacgcgtcccagtcaacgaagatctttctgttggcgacgataagaacgtctttgttattggcgatatgatgtcgctcaacaggcttccgggagtcgcacaggtagcaatccaaggcggtgaatacgttgctgagcagattgctgctggggtcgagggacgttcctcgtctgaacgcccagcctttgaatactacgacaagggttcgatggctacggtgtcgcgctttaacgccgttgtgaagcttggaaaagttgaagttacgggatttattggctgggtcatgtggcttctcgtccacttgatgttcttggtcggcttccgcaaccgagcaactgcggctttctcttggggcatcaatgcgctttcacgtaagcgttggaacctcgccaccactcgtcagcagcttcacggccgtactggtttgcaaaaacttactgcgctcgtcgataccgccgaaaagaag

SEQ ID NO: 6 - protein, 454 aa, Corynebacterium diphtheriae
MTNTPFRPEGGRHHVVVIGSGFGGLFAVQNLKDADVDITLIDRTNHHLFQPLLYQVATGILSSGEIAPQTRQVLAQQNNVHVLKAEVTDIDTESKTVVADLDDYSKTIEYDSLIVAAGAGQSYFGNDHFAEFAPGMKTIDDALELRARIIGAFERAEMCEDPKERERLLTFVIVGAGPTGVELAGQLAEMAHRTLSGEYTQFTPSNAKIILLDGAPQVLPPFGKRLGRTAQRELEKIGVTVKLNAIVTGVDENSVTYKSTVDDSLHTIDSFCKIWSAGVAASPLGKLVAEQLGVEVDRAGRVPVNEDLSVGDDKNVFVIGDMMSLNRLPGVAQVAIQGGEYVAEQIAAGVEGRSSSERPAFEYYDKGSMATVSRFNAVVKLGKVEVTGFIGWVMWLLVHLMFLVGFRNRATAAFSWGINALSRKRWNLATTRQQLHGRTGLQKLTALVDTAEKK

SEQ ID NO: 7 - DNA, 1302 bp, Escherichia coli
  CDS
ttgactacgccattgaaaaagattgtgattgtcggcggcggtgctggtgggctggaaatggcaacacagctggggcataagctgggacgcaagaaaaaagccaaaattacgctggtcgatcgtaaccacagccacctgtggaaaccgctgctgcacgaagtggcgactggctcgcttgatgaaggcgtcgatgcgttgagctatctggcccatgcgcgcaatcatggtttccagttccagctgggttccgtcattgatattgatcgtgaagcgaaaacaatcactattgcagaactgcgcgacgagaaaggtgaactgctggttccggaacgtaaaatcgcctatgacaccctggtaatggcgctgggtagcacctctaacgatttcaatacgccaggtgtcaaagagaactgcattttcctcgataacccgcaccaggcgcgtcgcttccaccaggagatgctgaatttgttcctgaaatactccgccaacctgggcgcgaatggcaaagtgaacattgcgattgtcggcggcggcgcgacgggtgtagaactctccgctgaattgcacaacgcggtcaagcaactgcacagctacggttacaaaggcctgaccaacgaagccctgaacgtaacgctggtagaagcgggagaacgtattttgcctgcgttaccgccacgtatctctgctgcggcccacaacgagctaacgaaacttggcgttcgcgtgctgacgcaaaccatggtcaccagtgctgatgaaggcggcctgcacactaaagatggcgaatatattgaggctgatctgatggtatgggcagccgggatcaaagcgccagacttcctgaaagatatcggtggtcttgaaactaaccgtatcaaccagctggtggtggaaccgacgctgcaaaccacccgcgatccagacatttacgctattggcgactgcgcgtcatgcccgcgtccggaagggggctttgttccgccgcgtgctcaggctgcacaccagatggcgacttgcgcaatgaacaacattctggcgcagatgaacggtaagccgctgaaaaattatcagtataaagatcatggttcgctggtatcgctgtcgaacttctccaccgtcggtagcctgatgggtaacctgacgcgcggctcaatgatgattgaaggacgaattgcgcgctttgtatatatctcgctataccgaatgcatcagattgcgctgcatggttactttaaaaccggattaatgatgctggtggggagtattaaccgcgttatccgtccgcgtttgaagttgcat

SEQ ID NO: 8 - protein, 434 aa, Escherichia coli
MTTPLKKIVIVGGGAGGLEMATQLGHKLGRKKKAKITLVDRNHSHLWKPLLHEVATGSLDEGVDALSYLAHARNHGFQFQLGSVIDIDREAKTITIAELRDEKGELLVPERKIAYDTLVMALGSTSNDFNTPGVKENCIFLDNPHQARRFHQEMLNLFLKYSANLGANGKVNIAIVGGGATGVELSAELHNAVKQLHSYGYKGLTNEALNVTLVEAGERILPALPPRISAAAHNELTKLGVRVLTQTMVTSADEGGLHTKDGEYIEADLMVWAAGIKAPDFLKDIGGLETNRINQLVVEPTLQTTRDPDIYAIGDCASCPRPEGGFVPPRAQAAHQMATCAMNNILAQMNGKPLKNYQYKDHGSLVSLSNFSTVGSLMGNLTRGSMMIEGRIARFVYISLYRMHQIALHGYFKTGLMMLVGSINRVIRPRLKLH

SEQ ID NO: 9 - DNA, 1296 bp, Pseudomonas fluorescens
  CDS
atgactcatcgtattgtcatcgttggcggcggcgccggcggtctggagttggctacccgtctgggtaagactctgggcaagcgtggcacggccagtgtgatgctggtcgacgcgaacctgacccacatctggaaaccgctactgcacgaagtggccgccggctccttgaactcctccgaagacgaactcaactatgtcgcccaggcaaaatggaaccacttcgagttccagctcgggcgcatgagcggcctggatcgcgagcgcaagagaatccaactggccgccacctatgacgagaccggcgtcgagctgttgccggcccgggaactgggctacgacaccctggtgattgccgtcggcagcaccaccaatgacttcggcaccgaaggcgcggcgcagcactgcctgttcctcgacacccgcaaacaggccgagcgcttccatcagcaattgctgcaccactatctgcgcgcccacgccgggcagaccgatatcgtcgagcgcatcagcgtcgccattgtcggcgccggtgcgaccggggtcgaactggccgccgagctgcataatgccgcccacgaactgcacgcctacggcctggaccggatcaaaccggagaacatgcacatcaccctgatcgaggccgggccacgcgtcttgccggccctgccggagcgtatcggcgggccggtgcacaagaccctggaaaaactcggggtcaacgtcatgaccaacgccgccgtcagccaggtgaccgccgacagcctgattaccgcggacggcaaagtgatcgacgcgagcctgaaagtctgggccgccgggattcgcgccccggacttcctcaaggacatcgacgggctggagaccaaccggatcaaccagttgcacgtgctgcccactttgcagaccacccgcgacgagaacatcttcgccttcggcgactgcgccgcctgcccgcaacccggcagcgagcgcaacgtcccacctcgcgcccaggccgcacaccagcaagcctcactgctggccaaatccttgaagctgcggatcgagggcaaggccctgccggaatacaaatacaccgactacggctcgctgatctcgctgtcgcggttctcggcagtgggcaacctgatgggtaacctgaccggcagcgtgatgctcgaaggctggctggcgcggatgttctatgtgtcgctgtaccgcatgcaccagatggcgctgtacggcatgttccgcacggccatgttgatgctgggtagcaagatcgggcgtgggaccgagcctcggctgaagctgcac

SEQ ID NO: 10 - protein, 432 aa, Pseudomonas fluorescens
MTHRIVIVGGGAGGLELATRLGKTLGKRGTASVMLVDANLTHIWKPLLHEVAAGSLNSSEDELNYVAQAKWNHFEFQLGRMSGLDRERKRIQLAATYDETGVELLPARELGYDTLVIAVGSTTNDFGTEGAAQHCLFLDTRKQAERFHQQLLHHYLRAHAGQTDIVERISVAIVGAGATGVELAAELHNAAHELHAYGLDRIKPENMHITLIEAGPRVLPALPERIGGPVHKTLEKLGVNVMTNAAVSQVTADSLITADGKVIDASLKVWAAGIRAPDFLKDIDGLETNRINQLHVLPTLQTTRDENIFAFGDCAACPQPGSERNVPPRAQAAHQQASLLAKSLKLRIEGKALPEYKYTDYGSLISLSRFSAVGNLMGNLTGSVMLEGWLARMFYVSLYRMHQMALYGMFRTAMLMLGSKIGRGTEPRLKLH

SEQ ID NO: 11 - DNA, 1296 bp, Azotobacter vinelandii
  CDS
atgactcatcgtatcgtaatcgtcggcggtggcgctggcggcgtggaactcgctacccgcctcggcaagaccatgggcaggaacttccaggcgaagatcaccctggtcgacgccaacatgacccacctgtggaaaccgctgctgcacgaagtcgccgccggctcgctgaactcgaccggcgacgaactgaactatgtggcccaggccaaatggaacaacttcgagttccagtacggccgcatgtgcggtctggaccgggccaacaagcgtatccgcctggcggcccagccggcccaggaagatcgcgcgcccctgcccgagcgcgaactggaatacgacaccctggtcctttccgtcggcagcaccaccaatgacttcggcaccccgggcgccgccgagaactgtattttcctggaaggccgcgaccaggccgagcgtttccgccgtccgctgctcagccactacttgcgtgcccacgccagcaatgacgacggccatcaggtcaaggtcgccatcgtcggcgccggtgccaccggggtcgaactggccgcagaactgcgccacgcctccaaggaactggtcgcctatgggctggagcgcattccgccggagaacctcagcatcacgctgatcgaatccagcccgcgtgtactcgccgccctgcccgaacgcatcagccgctccgcgcacgccaccctggaaagcctgggcgttcgcgtgctcgtcagcaccgccgtcagcgaggtcaccgcggaaggcgtgaagacgaaggacgaccagttcatccccgccgacctcatggtctgggccgcaggcgtccgcgcgcccgccttcctcaaggagctggatggtctggaaaccaatcgcatcaaccagttgcaggtccgccagaccctgcagactactctggacgacgatatcttcgccttcggcgattgtgcctcctgcccgcagccgggtaccgaccgccccgttccgccgcgcgcccaggccgctcaccagcaggccagcctgctggccaagtcgctccaccgcaagctgcaggaagacagcctctgctggagtatcgctacagcgaaccacggctcgctgatctccctctcgagcttctcggcgatcggtaacctgatgggcaacctgaccggcaacgtgaccttggaaggctggctggcccgcaagttctacatttccctgtaccgcatgcaccagatggcgctctacggtaccttccgcaccctgatgatgatgctgggcgaccgcttccgcagcagcaccgaaccccgcctcaagcttcac

SEQ ID NO: 12 - protein, 432 aa, Azotobacter vinelandii
MTHRIVIVGGGAGGVELATRLGKTMGRNFQAKITLVDANMTHLWKPLLHEVAAGSLNSTGDELNYVAQAKWNNFEFQYGRMCGLDRANKRIRLAAQPAQEDRAPLPERELEYDTLVLSVGSTTNDFGTPGAAENCIFLEGRDQAERFRRPLLSHYLRAHASNDDGHQVKVAIVGAGATGVELAAELRHASKELVAYGLERIPPENLSITLIESSPRVLAALPERISRSAHATLESLGVRVLVSTAVSEVTAEGVKTKDDQFIPADLMVWAAGVRAPAFLKELDGLETNRINQLQVRQTLQTTLDDDIFAFGDCASCPQPGTDRPVPPRAQAAHQQASLLAKSLHRKLQEDSLCWSIATANHGSLISLSSFSAIGNLMGNLTGNVTLEGWLARKFYISLYRMHQMALYGTFRTLMMMLGDRFRSSTEPRLKLH

SEQ ID NO: 13 - DNA, 1302 bp, Salmonella typhimurium LT2
  CDS
ttgactacaccattaaaaaagatcgtgattgtcggcggcggcgctggcgggctggaaatggcgacgcagttaggccataaactggggcgcaagaaaaaagcgaaaatcacgctggtagacagaaatcacagccatctgtggaaaccattgctgcacgaagtggcgactggctctctggacgaaggcgtggatgcgctgagctatctggctcatgcgcgtaatcatggtttccagttccagctggggtcggtgatggatatcgatcgcgaagcgaaaaccatcaccattgccgagttgcgtgatgaaaagggcgaactgctggtgccggagcgcaaaatcgcgtatgacacgctggtgatggcgctgggcagcacctctaatgatttcaacacgccgggcgtgaaagagcactgtatcttcctcgataacccgcatcaggcgcgccgttttcaccaggagatgctgaacctgttcctcaagtattcggctaatctgggcgcgaacggtaaggtcaatatcgccatcgttggcggcggtgcgacgggggttgagctgtcggctgaactgcataatgcggtaaaacagctgcatagctatggttataaggggctgaccaacgacgcgctgaacgtgacgttggttgaggctggcgagcgtattctgcctgcgttgccgccgcgtatctccagcgccgcgcataatgaactgaccaaactgggcgttcgcgtgctgacgcaaactatggtcaccagcgccgacgaaggcggtctgcatacgaaagaaggtgaatatattcaggccgatctgatggtgtgggcggcgggtatcaaagcgccagattttatgaaagagattggtggtctggaaacgaaccgcattaaccaactggtggtggaaccgacgctgcaaaccacgcgcgatcctgatatttatgcgattggcgactgcgcttcctgcgcacgtccggagggaggttttgtgccgcctcgcgctcaggcggcgcatcagatggctacctgcgcgatgaaaaacattctggcgcagatgaatggcaaaccgctgaaagcttaccagtataaagatcatggatcgctggtctctctgtccaacttctctaccgtgggtagtctgatggggaacctgacccgcggttcaatgatgattgaaggacgtatcgcccgcttcgtgtatatctcgctataccgtatgcaccagatagcgctgcatggatactttaaaaccggcctgatgatgctggtgggcagtattaaccgcgtcattcgtccgcgcctgaaactgcat

SEQ ID NO: 14 - protein, 434 aa, Salmonella typhimurium LT2
MTTPLKKIVIVGGGAGGLEMATQLGHKLGRKKKAKITLVDRNHSHLWKPLLHEVATGSLDEGVDALSYLAHARNHGFQFQLGSVMDIDREAKTITIAELRDEKGELLVPERKIAYDTLVMALGSTSNDFNTPGVKEHCIFLDNPHQARRFHQEMLNLFLKYSANLGANGKVNIAIVGGGATGVELSAELHNAVKQLHSYGYKGLTNDALNVTLVEAGERILPALPPRISSAAHNELTKLGVRVLTQTMVTSADEGGLHTKEGEYIQADLMVWAAGIKAPDFMKEIGGLETNRINQLVVEPTLQTTRDPDIYAIGDCASCARPEGGFVPPRAQAAHQMATCAMKNILAQMNGKPLKAYQYKDHGSLVSLSNFSTVGSLMGNLTRGSMMIEGRIARFVYISLYRMHQIALHGYFKTGLMMLVGSINRVIRPRLKLH

SEQ ID NO: 15 - DNA, 1908 bp, Lactobacillus plantarum WCFS1
  CDS
atggcaaagaaaaatattgtcgttgtcggtgcggggtttgctggtgtttacgcaaccaagaaactgtctaagcatttcaaaaaaaatgcagacgtcgagattacgttgattgaccggcattcatacttcacgtatatgactgaattacatgaagttgctaccgaacgggtggaacctgagcatatccaatatgatttgcaacggttgttcgcacggcgaaaaaacgttcgtctcgtgaccgataccgtgacgggcatcgacaaaaaggcacaaacagttactaccgaacacggaagttatcaatatgatcaacttttaattagtttgggtggggaatccaatgactttgggactcccggtgttaaggaacacggcttcgaattgtggtccttcgaacaagcgatggcattgcgcgctcacttatctgcaattattcggcggggggcggcggagctcgaccctgctaagcgcaaagccatgttgacctttacagtctgtggttctggttttactggttctgaactgattggtgaattaatcgaatatcgtgatgttttggctcgagacaacaagctcgatccaagtgaaatcacgctccaattggtcgaagcagcgccgactattattaacatgctcaaccggacgcaagccggtaaggccgctaagtacatggaaaaacatggtgtcaaaatcatgacgaactccatgattaccgaagtctgtgaagaccatgttaacttaaaaggcaaggatccaattccaacctacacgttaatctggacagccggtgttcgtgctaatagtatcgttaaaaagttcggcattgaaactaacccccgcggtggtcgcttgatggccaatgaattcatgcaagctaaggattgtaacaatatcttcttagccggtgattcaaccagctaccaagaacctgaccaaccacggccagtcccacaaatcgttcaaggggctgaagaaaccgcagctaaggccgtcgaaggtattattaagaacgttgaccagactgacgttacgatcaagccatttaagggcgcttatcaagcatccgtcgactcaattggttccaaatatgccgttgcacaagttttagagaagtggaacgtctctggttttattgccgtgcttttaaaacacgccatcaactggatgtactacgttcagattttctcaggttactacctattccagtacttcatgcacgaattcttccggactcgtaataaccgtaacgtcttccgcggttgggtctcacgggctggtaacgtactctggagtgtgccactgcggttcttctatggtgccatgtggttatgggactgctggactaaagttcagggatctgaatcctggttcactgacaagttacggttaccattcgaatggattaccgtggccgcaaccagtggtgcctctcaagcaactaaagccgctgcaaccagtggtgcttctgaagctgccacgtcaaccgttaaagccgctaagggtgtcttcagtctttcatacatgtacggtaaagaacccctgatggtctttgacaaaatgccacattggttcgaatcaattaccaaggtcttcattccgaacatgcaaatggccctcttcttccagaaattcatgacttgtgttgaaatcgtcattgcattatgtattttcttcggactctttacttggtttgctaacgcagtcaccattggcttagttgtcgtcttctgcttatctggcatgttctactgggtcaacatttggatgatctttgttgccctcgccttaatgaacggttccggacggacatttgggttagactactgggtcgttccgtggatgcaaaaacaccttggacactggtggtacggcaacgttcgttctcattacgacggtgttaaaacccgc

SEQ ID NO: 16 - protein, 636 aa, Lactobacillus plantarum WCFS1
MAKKNIVVVGAGFAGVYATKKLSKHFKKNADVEITLIDRHSYFTYMTELHEVATERVEPEHIQYDLQRLFARRKNVRLVTDTVTGIDKKAQTVTTEHGSYQYDQLLISLGGESNDFGTPGVKEHGFELWSFEQAMALRAHLSAIIRRGAAELDPAKRKAMLTFTVCGSGFTGSELIGELIEYRDVLARDNKLDPSEITLQLVEAAPTIINMLNRTQAGKAAKYMEKHGVKIMTNSMITEVCEDHVNLKGKDPIPTYTLIWTAGVRANSIVKKFGIETNPRGGRLMANEFMQAKDCNNIFLAGDSTSYQEPDQPRPVPQIVQGAEETAAKAVEGIIKNVDQTDVTIKPFKGAYQASVDSIGSKYAVAQVLEKWNVSGFIAVLLKHAINWMYYVQIFSGYYLFQYFMHEFFRTRNNRNVFRGWVSRAGNVLWSVPLRFFYGAMWLWDCWTKVQGSESWFTDKLRLPFEWITVAATSGASQATKAAATSGASEAATSTVKAAKGVFSLSYMYGKEPLMVFDKMPHWFESITKVFIPNMQMALFFQKFMTCVEIVIALCIFFGLFTWFANAVTIGLVVVFCLSGMFYWVNIWMIFVALALMNGSGRTFGLDYWVVPWMQKHLGHWWYGNVRSHYDGVKTR